(19) United States
(12) Patent Application Publication
(10) Pub. No.: US 2025/0386153 A1
(43) Pub. Date: Dec. 18, 2025

(54) ACQUISITION SEQUENCES OF BINAURAL HEARING SYSTEMS COMPRISING HEARING IMPLANTS

(71) Applicant: GN Hearing A/S, Ballerup (DK)

(72) Inventor: Michael Løngaa, Farum (DK)

(73) Assignee: GN Hearing A/S, Ballerup (DK)

(21) Appl. No.: 19/313,154

(22) Filed: Aug. 28, 2025

Related U.S. Application Data
(63) Continuation of application No. PCT/EP2024/058119, filed on Mar. 26, 2024.

(30) Foreign Application Priority Data
Mar. 31, 2023 (EP) 23165778.4

(51) Int. Cl. H04R 25/00

(52) U.S. Cl. CPC H04R 25/554; H04R 2225/67

(57) ABSTRACT

The present disclosure relates to acquisition sequences of binaural hearing systems and corresponding binaural hearing systems. The binaural hearing systems may comprise first and second hearing devices, for example head-wearable devices at a user's ears, and first and second hearing implants.

### RELATED APPLICATION DATA

This application is a continuation of International Patent Application No. PCT/EP2024/058119 filed on Mar. 26, 2024, which claims priority to, and the benefit of, European Patent Application No. 23165778.4 filed on Mar. 31, 2023, the entire disclosure of the above applications are expressly incorporated by reference herein.

### FIELD

The present disclosure relates to acquisition sequences of binaural hearing systems and corresponding binaural hearing systems. The binaural hearing systems may comprise first and second hearing devices, for example head-wearable hearing devices at a user's ears, and first and second hearing implants.

### BACKGROUND

Binaural hearing systems that comprise a pair of cochlea hearing implants may comprise at least four separate devices that are connected via at least three bidirectional wireless communication links during normal operation. A first bidirectional wireless communication link must be connected between a first hearing device, typically arranged at, in or on a user's ear, and a first hearing implant such as a cochlea implant. A second bidirectional wireless communication link must be connected between a second hearing device, typically arranged at, in or on the user's opposite ear, and a second hearing implant. Finally, a third bidirectional wireless communication link must be connected between the first and second hearing devices, i.e. a bilateral link, to allow the hearing devices arranged on opposite sides of the user's head to communicate data for example digital audio signals to enable sophisticated binaural processing algorithms and/or synchronize functions of the first and second hearing devices.

The respective acquisitions of the first, second and third bidirectional wireless communication links during an acquisition sequence after power-on of the binaural hearing systems are often time consuming, in particular in an adverse electromagnetic noise environment. This situation will leave the user without audible sound, and thus without auditory awareness, for a prolonged period of time after he/she activates the binaural hearing system. This absence of audible sound will continue until the acquisition sequence is completed and normal operation of the binaural hearing system started. Hence, it would be advantageous to rapidly provide the user with audible sound via nerve stimuli by implanted electrode arrays of the first and second cochlea implants quickly after power-on of the binaural hearing system and before the acquisition sequence is completed.

### SUMMARY

A first aspect of the subject disclosure relates to a binaural hearing system comprising:
a first hearing device, a second hearing device, a first hearing implant and a second hearing implant; wherein
the first hearing device is connectable to the first hearing implant via a first bidirectional wireless communication link for exchange of first ipsilateral data packets. The second hearing device is connectable to the second hearing implant via a second bidirectional wireless communication link for exchange of second ipsilateral data packets. A bilateral bidirectional wireless communication link is connectable between the first hearing device and second hearing device for exchange of bilateral data packets. The bilateral bidirectional wireless communication link and said first and second bidirectional wireless communication links are configured to operate in accordance with a common communication protocol. The communication protocol may comprise a plurality of consecutive frames each comprising a plurality of time slots such as 4 or more time slots. The common communication protocol comprises an acquisition sequence which comprises:
acquire the first bidirectional wireless communication link using a first processing unit of the first hearing device and acquire the second bidirectional wireless communication link using a second processing unit of the second hearing device. The first and second processing units are configured to acquire the bilateral bidirectional wireless communication link in response to acquisitions of the first and second bidirectional wireless communication links.

After a successful completion of the acquisition sequence, the binaural hearing system preferably enters a normal operation mode where the first hearing device and the first hearing implant are connected, such as wirelessly connected, through the first bidirectional wireless communication link. The second hearing device and the second hearing implant are likewise connected, such as wirelessly connected, connected through the second bidirectional wireless communication link during normal operation. Finally, the first hearing device and the second hearing device are connected, such as wirelessly connected, through the bilateral bidirectional wireless communication link during normal operation. The common communication protocol may be proprietary and designed for minimal power consumption since each of the first and second hearing devices and each of the first and second hearing implants typically are relatively small battery powered devices or otherwise energized by a power source with limited capacity.

One of the first hearing device and second hearing device is preferably configured as master and the other one as slave for the purpose of acquiring and operating the bilateral bidirectional wireless communication link using their respective first and second processing units as discussed in additional detail below with reference to the appended drawings.

Each of the first and second hearing devices may comprise a head-wearable housing shaped and sized similarly to a traditional hearing aid for example of so-called BTE, ITE, ITC, CIC or RIC types of housings. The housings may be shaped and sized for placement at, or in, a user's left or right ear, for example shaped and sized for placement behind the user's left and right ear pinna. The first and second hearing implants may be configured for placement at the respective sides of the user's skull and configured to supply respective nerve stimulus signals to the user's left and right hearing nerves via implanted electrode arrays.

Each of the first and second bidirectional wireless communication links and the bilateral bidirectional wireless communication link may be based on near-field magnetic coupling, such as NFMI, using respective magnetic coil antennas mounted in the first and second hearing devices. Each of the first and second bidirectional wireless communication links and the bilateral bidirectional wireless communication link may for example use a carrier frequency between 5 and 50 MHz as discussed in additional detail below with reference to the appended drawings.

Each of the first processing unit and second processing unit may comprise a digital signal processor (DSP) and/or a microprocessor such as a software programmable DSP or a software programmable microprocessor. The software programmable DSP or microprocessor may be configured to execute plurality of program instructions configured to implement at least parts of the respective acquisitions of the first and second bidirectional wireless communication links and the bilateral bidirectional wireless communication link in accordance with the common communication protocol. Each of the first processing unit and second processing unit may comprise a dedicated digital state machine configured to handle certain steps of the acquisitions of the first and second bidirectional wireless communication links and the bilateral bidirectional wireless communication link in accordance with the common communication protocol.

According to an embodiment of the common communication protocol, and preferably a corresponding embodiment of the binaural hearing system, the acquisition sequence comprises:
maintain at least one of the first bidirectional wireless communication links and second bidirectional wireless communication links during acquisition of the bilateral bidirectional wireless communication link. The maintenance of at least one of the first and second bidirectional wireless communication links during acquisition of the bilateral bidirectional wireless communication link provides the user with sound and general auditory awareness at one side of the user's head until the acquisition sequence is completed and the normal operation of the binaural hearing system may be commenced as discussed in further detail below with reference to the appended drawings.

According to an embodiment of the common communication protocol, and preferably a corresponding embodiment of the binaural hearing system, the acquisition of the first bidirectional wireless communication link comprises:
transmit a first synchronization marker from the first processing unit to the first hearing implant in a first time slot at the first processing unit,
monitor the plurality of time slots, at the first hearing implant for the first synchronization marker,
sliding the plurality of time slots at the first hearing implant with predetermined time steps between frames of the plurality of consecutive frames using a wrap-around scheme,
detect the first synchronization marker at the first hearing implant,
transmit an acknowledge message from the first hearing implant to the first processing unit in second time slot at the first hearing implant,
synchronize the plurality of time slots at the first hearing implant with the plurality of time slots at the first processing unit based on the first synchronization marker,
complete the acquisition of the first bidirectional wireless communication link; and
the acquisition of the second bidirectional wireless communication link comprises:
transmit a second synchronization marker from the second processing unit to the second hearing implant in a first time slot at the second processing unit,
monitor the plurality of time slots, at the second hearing implant for the second synchronization marker,
sliding the plurality of time slots at the second hearing implant with predetermined time steps between frames of the plurality of consecutive frames using a wrap-around scheme,
detect the second synchronization marker at the second hearing implant,
transmit an acknowledge message from the second hearing implant to the second processing unit in a second time slot at the second hearing implant,
synchronize the plurality of time slots at the second hearing implant with the plurality of time slots at the second processing unit based on the second synchronization marker,
complete the acquisition of the second bidirectional wireless communication link.

According to an embodiment of the binaural hearing system, the acquisition sequence comprises:
exchange the first ipsilateral data packets using first and second time slots at the first processing unit of respective frames of the plurality of consecutive frames,
exchange the second ipsilateral data packets using first and second time slots at the second processing unit of respective frames of the plurality of consecutive frames,
transmit a third synchronization marker from the first processing unit to the second processing unit in a third time slot at the first processing unit,
detect the third synchronization marker at the second processing unit,
synchronize the plurality of time slots at the second processing unit with the plurality of time slots at the first processing unit based on the third synchronization marker,
terminate/complete the acquisition sequence,
enter normal operation of the binaural hearing system.

The common communication protocol may comprise plurality of time slots of each frame such as at least four time slots that enable the first and second hearing devices to exchange the bilateral data packets in the first and second time slots. The plurality of time slots may be non-overlapping time slots. Each of the at least four non-overlapping time slots may have a length between 24 μs and 384 μs. The respective lengths, i.e. duration in time, of the at least four non-overlapping time slots may be identical.

In the first time slot the first processing unit may transmit a bilateral data packet to the second hearing device via the bilateral bidirectional wireless communication link. In a corresponding manner the second hearing device may transmit another bilateral data packet to the first processing unit using the second time slot via the bilateral bidirectional wireless communication link. In the third time slot, each of the first and second processing units may transmit one of the first ipsilateral data packets to their respective hearing implants. In a fourth time slot, each of the first and second hearing implants may transmit one of the second ipsilateral data packets to their respective hearing devices.

In some embodiments of the binaural hearing system at least a subset of the first and second ipsilateral data packets comprises respective digital audio signals or audio data such as real-time digital audio signals and/or at least a subset of the bilateral data packets comprises respective digital audio signals or data. The respective subsets of the first and second ipsilateral data packets allow the first and second hearing devices to transmit processed sound to the first and second hearing implants. The digital audio signals may, for example, be derived from respective microphone arrangements integrated in housings of the first and second hearing devices.

In one embodiment of the common communication protocol and corresponding binaural hearing system, the acquisition of the bilateral bidirectional wireless communication link comprises:
shift transmission of the second ipsilateral data packets at the second processing unit with one time slot between frames of the plurality of consecutive frames using wrap-around,
shift transmission of the third synchronization marker with a predetermined time step through the third time slot using wrap-around at the first processing unit.

According to one embodiment of the binaural hearing system the predetermined time step by which each of the first, second and third synchronization marker is shifted or slided less than 5% of a length of one time slot of the plurality of time slots of the frame.

According to one embodiment of the binaural hearing system the second bidirectional wireless communication link is temporarily broken during the acquisition of the bilateral bidirectional wireless communication link while the first bidirectional wireless communication link is maintained during the acquisition of the bilateral bidirectional wireless communication link. In this embodiment, the acquisition of the bilateral bidirectional wireless communication link may comprise:
monitor, at the second processing unit, the bidirectional wireless communication link for the second synchronization marker,
temporarily break the second bidirectional wireless communication link by the second processing unit,
interrupt the shift of time slots by the second processing unit while maintaining the transmission shift of the synchronization marker by the first processing unit,
detect by the first processing unit when the second synchronization marker is arranged within the third time slot at the first processing unit,
detect the second synchronization marker at the second processing unit and transmit an acknowledge message by the second processing unit for example as response,
re-acquire the second bidirectional wireless communication link before the completion, e.g. termination, of the acquisition sequence.

In one embodiment of the binaural hearing system the acquisition sequence comprises:
maintain connection through of the first bidirectional wireless communication link and connection through the second bidirectional wireless communication link during acquisition of the bilateral bidirectional wireless communication link. Since the respective connections of the first and second bidirectional wireless communication links are established, the exchange of the first ipsilateral data packets and the exchange of the second ipsilateral data packets may provide the user of the binaural hearing system with sound at both ears during the subsequent acquisition of the bilateral bidirectional wireless communication link. That feature is desirable for numerous reasons such as providing the user with auditory awareness as discussed in further detail below with reference to the appended drawings.

One embodiment of the binaural hearing system, where the connections through both of the first and second bidirectional wireless communication links are maintained during the acquisition of the bilateral bidirectional wireless communication link, comprises:
shift transmission of the second ipsilateral data packets at the second processing unit by a predetermined time step between frames of the plurality of consecutive frames using wrap-around. The predetermined time step is preferably much shorter than a length of one time slot for example less than 5% or less than 1% length of one time slot. The first processing unit monitors the bilateral bidirectional wireless communication link for an acknowledge message transmitted by the second processing unit in response to detection of the third synchronization marker at the second processing unit.

The time step shift of the transmission of the second ipsilateral data packets may comprise:
advancing the transmission for one or more round trips in a first time direction through the plurality of time slots,
temporarily interrupt the advancement of the transmission for one or more round trips,
restart the advancement of transmission in a second time direction through the plurality of time slots for one or more round trips. The first and second time directions are opposite.
interrupt the advancement of the transmission by the second processing unit in response to transmission of the acknowledge message,
synchronize the plurality of time slots at the second processing unit with the plurality of time slots at the first processing unit based on the second synchronization marker.

The skilled person will understand that this opposite oriented time sliding or time stepping of the transmissions of the second ipsilateral data packets may serve to compensate for misaligned, i.e. unaligned, free-running clock generators and clock signals at the first and second processing units before the bilateral bidirectional wireless communication link is acquired.

According to one embodiment of the binaural hearing system the synchronization marker comprises a unique pair ID for pairing at least one of:
the first hearing device and the first hearing implant,
the first hearing device and the second hearing device; and
the second hearing device and the second hearing implant.

Certain embodiments of the binaural hearing system utilize three different unique pair IDs in the synchronization marker to avoid erroneous pairing of devices of the binaural hearing system as discussed in further detail below with reference to the appended drawings.

A second aspect of the subject disclosure relates to a binaural hearing system comprising:
a first hearing device connected to a first hearing implant via a first bidirectional wireless communication link for exchange of first ipsilateral data,
a second hearing device connected to a second hearing implant via a second bidirectional wireless communication link for exchange of second ipsilateral data,
the first hearing device connected to the second hearing device via a bilateral bidirectional wireless communication link connecting for exchange of bilateral data;
wherein
said bilateral bidirectional wireless communication link and said first and second bidirectional wireless communication links are configured to operate in accordance with a common communication protocol which comprises a plurality of consecutive frames each comprising a plurality of time slots;
said common communication protocol comprising an acquisition sequence which comprises:
acquire a first connection through the first bidirectional wireless communication link using a first processing unit of the first hearing device,
acquire a second connection through the second bidirectional wireless communication link using a second processing unit of the second hearing device, wherein the first processing unit is configured to acquire a third connection through the bilateral bidirectional wireless communication link in response to the acquisition by the first processing unit of the first hearing device of the first connection through the first bidirectional wireless communication link and in response to the acquisition by the second processing unit of the second hearing device of the second connection through the second bidirectional wireless communication links.

The content of each of the first, second and third synchronization markers may be defined by the common communication protocol and identical except for unique pair IDs as discussed in further detail below with reference of the appended drawings.

A third aspect of the subject disclosure relates to an acquisition sequence, e.g. a computer-implemented acquisition method, for acquiring respective connections, such as wireless connections, between a first hearing device and a first hearing implant, between a second hearing device and a second hearing implant and between the first hearing device and the second hearing device.

The acquisition sequence comprising:
acquire a first bidirectional wireless communication link between the first hearing device and the first hearing implant,
exchange first ipsilateral data packets through the first bidirectional wireless communication link,
acquire a second bidirectional wireless communication link between the second hearing device and the second hearing implant,
exchange second ipsilateral data packets through the second bidirectional wireless communication link; and in response to acquisitions of the first and second bidirectional wireless communication links:
acquire, in response to acquisitions of the first and second bidirectional wireless communication links, a bilateral bidirectional wireless communication link between the first and second hearing devices,
exchange bilateral data packets between the first hearing device and the second hearing device.

One embodiment of the acquisition sequence comprises:
exchange the first ipsilateral data packets using first and second time slots at the first processing unit of respective frames of the plurality of consecutive frames,
exchange the second ipsilateral data packets using first and second time slots at the second processing unit of respective frames of the plurality of consecutive frames,
transmit a third synchronization marker from the first processing unit to the second processing unit in a third time slot at the first processing unit,
detect the synchronization marker at the second processing unit,
synchronize the plurality of time slots at the first processing unit with the plurality of time slots at the second processing unit based on the third synchronization marker.

A fourth aspect of the subject disclosure relates to a binaural hearing system comprising:
a first hearing device, a second hearing device, a first hearing implant and a second hearing implant; and
a first synchronous connection between the first hearing device and the first hearing implant through a first wireless communication link for exchange of first ipsilateral data packets,
a second synchronous connection between the second hearing device and the second hearing implant through a second wireless communication link for exchange of second ipsilateral data packets,
a third synchronous connection between the first hearing device and the second hearing device through a third wireless communication link for exchange of bilateral data packets. The skilled person will understand that the first synchronous connection may be established by any of the acquisition sequences disclosed above in relation to the first, second and third aspects and/or by any of the acquisition sequences of the exemplary binaural hearing system disclosed below.
Likewise, may the second synchronous connection be established by any of the acquisition sequences disclosed above in relation to the first, second and third aspects and/or by any of the acquisition sequences of the exemplary binaural hearing system disclosed below.
Likewise may the third synchronous connection be established by any of the acquisition sequences disclosed above in relation to the first, second and third aspects and/or by any of the acquisition sequences of the exemplary binaural hearing system disclosed below.

Each of the first synchronous connection, the second synchronous connection and the third synchronous connection according to the fourth aspect may be configured to operate in accordance with a predetermined communication protocol such as the common communication protocol disclosed above in relation to the first, second and third aspects and/or to the common communication protocol utilised by any of the exemplary binaural hearing system disclosed below.

Further each of the first hearing device, second hearing device, first hearing implant and a second hearing implant may be similar or identical to the respective ones of the first hearing device, second hearing device, first hearing implant and a second hearing implant disclosed above in relation to the first, second and third aspects and/or the embodiments disclosed below in relation to the exemplary binaural hearing systems disclosed below. Each of the first hearing implant and second hearing implant may comprise at least 4 stimulating channels such as more than 8 stimulating channels or more than 16 stimulating channels configured to stimulate the user's cochlea nerve.

### BRIEF DESCRIPTION OF THE DRAWINGS

In the following exemplary embodiments are described in more detail with reference to the appended drawings, wherein:

FIG. 1 schematically illustrates an exemplary binaural hearing system mounted on user's head in accordance with some embodiments,

FIG. 2 shows respective schematic block diagrams of first and second hearing devices and associated first and second hearing implants of the exemplary binaural hearing system preferably operating in accordance with the common communication protocol,

FIG. 3 schematically illustrates transmission of a first synchronization marker from the first hearing device to the first hearing implant during acquisition of the first bidirectional wireless communication link according to the embodiments of the exemplary binaural hearing system,

FIG. 4 schematically illustrates detection of the first synchronization marker by the first hearing implant and its response by transmission of an acknowledge message during acquisition of the first bidirectional wireless communication link according to embodiments of the exemplary binaural hearing system,

FIG. 4A schematically illustrates exchange of respective ipsilateral data packets between the first hearing device and the first hearing implant and between the second hearing device and the second hearing implant following the acquisition of the first and second bidirectional wireless communication links in accordance with embodiments of the exemplary binaural hearing system,

FIG. 5 schematically illustrates exchange of respective ipsilateral data packets between the first hearing device and the first hearing implant and between the second hearing device and the second hearing implant during acquisition of a bilateral bidirectional wireless communication link in accordance with embodiments of the exemplary binaural hearing system such as a first embodiment thereof.

FIG. 6 schematically illustrates use of a sliding synchronization marker during the acquisition of the bilateral bidirectional wireless communication link in accordance with the embodiments of the exemplary binaural hearing system, such as the first embodiment,

FIG. 7 schematically illustrates the use of the sliding synchronization marker during the acquisition of the bilateral bidirectional wireless communication link in accordance with the embodiments of the exemplary binaural hearing system, such as the first embodiment,

FIG. 8 illustrates features of an exemplary synchronization marker utilized during the acquisition of the bilateral bidirectional wireless communication link in accordance with the embodiments of the exemplary binaural hearing system, such as the first and a second embodiment,

FIG. 9 illustrates exemplary super-frame structures each comprising a plurality of frames of the ipsilateral data packets and bilateral data packets in accordance with the embodiments of the exemplary binaural hearing system, such as the first and second embodiment,

FIG. 10 schematically illustrates exchange of ipsilateral data packets and bilateral data packets during normal operation, i.e. payload mode, of the exemplary binaural hearing system after completion of the acquisition sequence in accordance with the embodiments of the exemplary binaural hearing system, such as the first and second embodiments,

FIG. 11 schematically illustrates the exchange of respective ipsilateral data packets between the first hearing device and the first hearing implant and between the second hearing device and the second hearing implant during acquisition of a bilateral bidirectional wireless communication link in accordance with embodiments of the exemplary binaural hearing system, such as the second embodiment,

FIG. 12 schematically illustrates the exchange of respective ipsilateral data packets between the first hearing device and the first hearing implant and between the second hearing device and the second hearing implant during the acquisition of the bilateral bidirectional wireless communication link in accordance with the embodiments of the exemplary binaural hearing system, such as the second embodiment,

FIG. 13 schematically illustrates the exchange of respective ipsilateral data packets between the first hearing device and the first hearing implant and between the second hearing device and the second hearing implant during the acquisition of the bilateral bidirectional wireless communication link in accordance with the embodiments of the exemplary binaural hearing system, such as the second embodiment; and

FIG. 14 and FIG. 15 are flow charts illustrating respective steps carried out by the first and second processing units of the first and second hearing devices, respectively, during an acquisition sequence of the bilateral bidirectional wireless communication link and the first and second bidirectional wireless communication links according to the embodiments of the exemplary binaural hearing system, such as the second embodiment.

### DETAILED DESCRIPTION

Various embodiments are described hereinafter with reference to the figures. Like reference numerals refer to like elements throughout. Like elements will, thus, not be described in detail with respect to the description of each figure. It should also be noted that the figures are only intended to facilitate the description of the embodiments. They are not intended as an exhaustive description of the claimed invention or as a limitation on the scope of the claimed invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

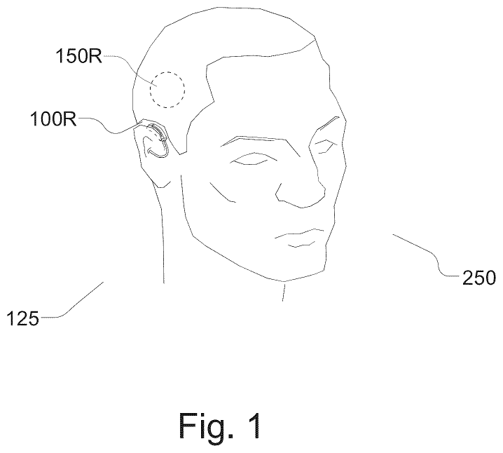

FIG. 1 schematically illustrates an exemplary binaural hearing system 125 mounted on a user's head 250. The exemplary binaural hearing system 125 comprises a second hearing device 100R and a second hearing implant 150R for example mounted on/in a right side of the user's head 250. The binaural hearing system 125 further comprises a first hearing device (not shown) and a first hearing implant (not shown) that are both arranged at the opposite side of the user's head 250 and therefore not visible on the drawing. The second hearing implant 150R and the first hearing implant are adapted for surgical implantation in opposite sides of the user's skull and are therefore invisible from the outside when implanted.

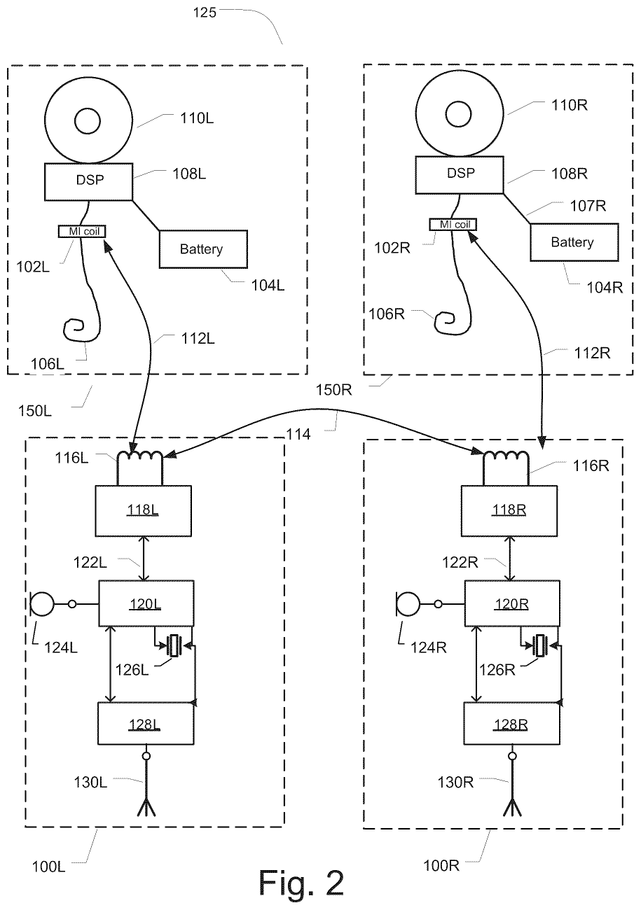

FIG. 2 is a block diagram of the exemplary binaural hearing system 125 such as the binaural hearing systems according to the exemplary embodiments, such as first and second embodiments thereof, disclosed below. The first hearing device 100L is connectable to the first hearing implant 150L via a first bidirectional wireless communication link 112L (“first link”) for exchange of first ipsilateral data packets. The second hearing device 100R is likewise connectable to the second hearing implant 150R via a second bidirectional wireless communication link 112R (“second link”) for exchange of second ipsilateral data packets.

The binaural hearing system 125 further comprises a bilateral bidirectional wireless communication link 114 (“bilateral link”, or “third link”) that is connectable between the first hearing device 100L and second hearing device 100R for exchange of bilateral data packets. The first hearing device 100L and second hearing device 100R are preferably configured to use the respective magnetic coil antennas 116L, 116R for wireless communication with the first hearing implant 150L and second hearing implant 150R, respectively.

The bilateral link 114, first link 112L and second link 112R are preferably configured to utilize, i.e. operate in accordance with, a common data communication protocol. The common data communication protocol preferably comprises a plurality of consecutive time frames that each comprises a plurality of time slots holding the first and second ipsilateral data packets and the bilateral data packets. The plurality of first and second ipsilateral data packets and bilateral data packets is structured, exchanged, e.g. transmitted and received, in accordance with the common data communication protocol as discussed in further detail below.

The skilled person will understand that certain types of control data packets such as synchronization markers and acknowledge messages may be wirelessly exchanged, i.e. transmitted and/or received, between the first hearing device 100L and the second hearing device 100R, between the first hearing device 100L and the first hearing implant 150L and between the second hearing device 100R and the second hearing implant 150R in connection with, e.g. before, the respective acquisitions of the bilateral link 114, first link 112L and second link 112R are completed.

The bilateral data packets exchanged through the bilateral link 114 may comprise digital audio signals and control information, e.g. in the control data packets, transmitted by respective ones of the first and second hearing devices 100L, 100R. Each of the first and second hearing devices 100L, 100R, respectively, may transmit digital audio samples and control data to the first and second hearing implant 150L, 150R, respectively. Each of the first and second hearing implants 150L, 150R may additionally be configured to transmit respective control data packets to the first and second hearing devices 100L, 100R, respectively, via the first link 112L and second link 112R. In the latter embodiment both the first link 112L and the second link 112R are adapted for bidirectional data transfer, such as transmission of the control data packets.

Each of the first link 112L, the second link 112R and the bilateral link 114 may in certain embodiments of the binaural hearing system 125 be based on, or comprise, near-field magnetic coupling, such as NFMI links. The first link 112L may accordingly comprise a first magnetic coil antenna 116L of the first hearing device 100L and a first magnetic coil antenna 102L of the first hearing implant 150L. The second link 112R may likewise comprise a second magnetic coil antenna 116R of the second hearing device 100R and a second magnetic coil antenna 102R of the second hearing implant 100R. Each of the first link 112L, the second link 112R and the bilateral link 114 may for example utilize a carrier frequency between 5 MHz and 50 MHZ, such as between 9 MHz and 27 MHz for respective transmissions of the first and second ipsilateral data packets and the bilateral data packets.

The first hearing implant 150L comprises a first control unit 108L, such as a first implant processor, that is connected to the first magnetic coil antenna 102L for receipt and processing of the first ipsilateral data packets transmitted by the first hearing device 100L. The first control unit 108L is further configured to transmit the first ipsilateral data packets to the first hearing device 100L via the first link 112L. In some embodiments, the first control unit 108L of the first hearing implant 150L may comprise a digital signal processor (DSP) and/or a microprocessor. Certain embodiments of the first hearing implant 150L may comprise a rechargeable battery assembly 104L. The first rechargeable battery assembly 104L is configured for receiving electrical power from a first receiver (Rx) charging coil 110L of the first hearing implant 150L during dedicated recharging operations or sessions. The first receiver (Rx) charging coil 110L may be energized by an appropriately positioned external, i.e. outside the user's skull, transmitter (Tx) charging coil (not shown) of a charging device (not shown) during the dedicated recharging sessions. This allows wireless transfer of power to the first rechargeable battery assembly 104L. The first rechargeable battery assembly 104L is preferably coupled to a power supply input of a first control unit 108L to energize the latter as schematically indicated by a first power line or wire 107L. The first control unit 108L may comprise a microprocessor for example software programmable microprocessor. The second hearing device 100R may comprise a similar second receiver (Rx) charging coil 110R and/or a similar second rechargeable battery assembly 104R and/or a similar second power line/wire 107R as schematically illustrated on FIG. 2.

The first hearing implant 150L further comprises a first electrode array 106L for insertion into a cochlea of the user during implantation. The first electrode array 106L may be electrically coupled to the first control unit 108L that supplies suitable electrode stimuli signals to the first electrode array 106L to stimulate the user's cochlea nerve. The skilled person will understand that these electrode stimuli signals may be generated by the first control unit 108L and derived by the latter based on the first ipsilateral data packets, in particular digital audio signals or data embedded or held in the first ipsilateral data packets. The skilled person will appreciate that corresponding functions, structures and features of the second hearing implant 150R may be largely identical to those of the first hearing implant 150L. The second hearing implant 150R may comprise a similar second electrode array 106R as schematically illustrated on FIG. 2.

The block diagram on FIG. 2 illustrates an exemplary embodiment of the first hearing device 100L. The first hearing device 100L comprises the first magnetic coil antenna 116L which is electrically connected to a first transceiver 118L. The first transceiver 118L is configured to repeatedly switch between Tx and Rx modes to modulate and demodulate incoming and outgoing data packets of the first ipsilateral data packets and to modulate and demodulate incoming and outgoing data packets of the bilateral data packets. The first transceiver 118L may be configured to convert the first ipsilateral data packets and bilateral data packets to a format understood by a first processing unit 120L of the first hearing device 100L. The first transceiver 118L may be electrically connected to the first processing unit 120L e.g. through a first data line or first data bus 122L for transmission of the first ipsilateral data packets and the bilateral data packets to the first processing unit 120L.

In some embodiments, the first processing unit 120L may comprise a digital signal processor (DSP) and/or a microprocessor such as a software programmable DSP or a microprocessor. The second hearing device 100R may comprise a similar second transceiver 118R connected in a similar manner to the second processing unit 120R through a second data line or second data bus 122R for transmission of the second ipsilateral data packets and transmission of the bilateral data packets to the second processing unit 120R as schematically illustrated on FIG. 2.

The first hearing device 100L further comprises one or more first microphones 124L coupled to an appropriate audio interface of the first processing unit 120L. The first processing unit 120L may be configured to execute a suitable operating system. The operating system may be configured to manage various hardware and software resources of the first hearing device 100L such as handling of the common communication protocol, computation of monaurally or bilaterally beamformed microphone signals, hearing loss compensation processing of first microphone signal(s), the first transceiver 118L, certain memory resources etc. The operating system may schedule tasks for efficient use of hearing device resources and may further include accounting software for cost allocation, including power consumption, processor time, memory locations, wireless transmissions and other resources. The operating system may be stored in and retrieved from a non-volatile memory (not shown), e.g. flash memory or EEPROM, of the first processing unit 120L. The second processing unit 120R of the second hearing device 100R may be configured in a corresponding manner to the first processing unit 120L. The second hearing device 100R may comprise one or more second microphones 124R coupled to an appropriate audio interface of the second processing unit 120R as schematically illustrated on FIG. 2.

Each of the first and second hearing devices 100L, 100R may comprise a housing of a type that is well-known from the hearing aid industry like so-called BTE, ITE, ITC, CIC or RIC housing types. These housing types are shaped and sized for placement at, or in, the user's ear. The first hearing device 100L further comprises a first system clock generator 126L that is configured to supply first clock signals to various digital logic circuits and components of the first hearing device 100L including the first processing unit 120L as schematically illustrated. A nominal value of a clock frequency of the first system clock generator 126L may lie between 2 MHz and 64 MHz such as between 10 MHz and 50 MHz. The first processing unit 120L may be configured to derive respective lengths of certain time slots and time frames of the common communication protocol of the first link 112L and the bilateral link 114 as discussed in additional detail below. The second hearing device 100R may comprise a similar second system clock generator 126R.

The first hearing device 100L may additionally comprise a second, optional, wireless communication interface 128L, such as a first radio interface, and a first RF antenna 130L configured to jointly communicate through a second wireless communication link, such as a first radio link (not shown). The first RF antenna 130L, the first radio link and the first radio interface 128L may be configured to operate in the 2.4 GHz industrial scientific medical (ISM) band. The first RF antenna 130L and the first radio interface 128 may be compliant with the Bluetooth LE standard. This first radio link may be configured to provide convenient data connectivity to various types of portable communication devices like smartphones, mobile phones, tablets and personal computers etc. due to the industry standard compatible nature of Bluetooth LE. Various types of control data and audio data may be transmitted from the portable communication device to the first hearing device 100L and vice versa. The second hearing device 100R may for the same purpose comprise a similar second, optional, wireless communication interface 128R, such as a second radio interface 128R (of the second hearing device 100R) and a second RF antenna 130R (of the second hearing device 100R) configured to jointly communicate through a second radio link as illustrated on FIG. 2.

In the following disclosure the first hearing device 100L of the exemplary binaural hearing system 125 is preferably assigned as master during execution of the common communication protocol while the second hearing device 100R is configured as a slave or vice versa. The skilled person will understand that the configuration of the first and second hearing devices 100L, 100R as master and slave may be carried out in connection with manufacturing of the binaural hearing system 125. Alternatively, the configuration of the first and second hearing devices 100L, 100R as master and slave may be carried out in connection with a fitting procedure of the binaural hearing system 125 on the user for example by utilizing an appropriately programmed computer connected to the binaural hearing system 125.

Hence, the first processing unit 120L of the first hearing device 100L may be configured as a master processing unit 120L and the second processing unit 120R may be configured as a slave processing unit for the acquisition of the bilateral link 114 as described in the below-disclosed exemplary embodiments of the binaural hearing system 125. The skilled person will understand that system clock signal, such as the first clock signal, of the first hearing device 100L when configured as the master may control timing of transmissions of the respective data packets through the first link 112L, the second link 112R and the bilateral link 114 at least after the respective acquisitions of the first link 112L, second link 112R and bilateral link 114.

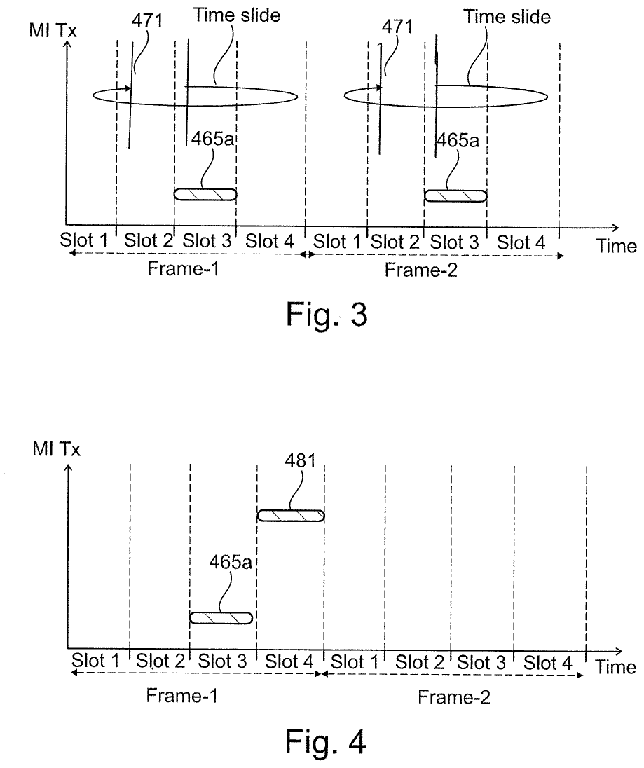

FIG. 3 schematically illustrates transmission of a first synchronization marker 465a from the first processing unit 120L o to the first hearing implant 150L during acquisition of the first link 112L according to embodiments of the common communication protocol of the exemplary binaural hearing system 125.

A “time slot” is the shortest time division of the bilateral bidirectional wireless communication link 114 as defined by the common communication protocol. The time slot of each of the first and second bidirectional wireless communication links is the shortest time division thereof as defined by the common communication protocol. One data packet may for example be transmitted in one time slot.

A “frame” is a unit of data that comprises a predefined number of time slots as defined by the common communication protocol.

“Connectable” shall mean that the wireless communication link in question is configured to establish a connection, such as wireless connection, between specified devices after acquisition of the wireless communication link in question.

A “bidirectional wireless communication link” is a wireless communication link that supports transmission of data packets or data messages from a first device to a second device and transmission of data packets or data messages from the second device to the first device.

“Ipsilateral data packets” are data packets exchanged between a hearing device and a hearing implant arranged at the same side of a user's head.

A “hearing implant” shall mean that portion of a cochlear implant that is located inside the user's skull.

During the acquisition of the first link 112L the first processing unit 120L operates as a master device towards the first hearing implant 150L. The first processing unit 120L may start to repeatedly transmit the first synchronization marker 465a to the first hearing implant 150L using one of a plurality of time slots, e.g. time slots 1-4, of each frame, Frame-1, Frame-2 etc., of a plurality of consecutive frames. The indicated time slots 1-4 are referenced to the first processing unit 120L. Time slot 3, at the first processing unit 120L, is selected for transmission of the first synchronization marker 465a in the present example as illustrated and made in accordance with the common communication protocol. The first hearing implant 150L resides concurrently in a receipt mode such that the first hearing implant 150L monitors the first link 112L to detect arrival of the first synchronization marker 465a. However, the respective time slots 1-4 at the first processing unit 120L and the first hearing implant 150L are most likely more or less misaligned or asynchronous at the current step of the acquisition sequence. This lack of alignment is caused by free-running first clock generators 126L and first clock signals at the first processing unit 120L and at the first hearing implant 150L. As illustrated by FIG. 3, where the first hearing implant 150L searches for a first synchronization marker 465a, which preferably comprises a first unique pair ID as discussed further below, in a time slot 471 at the first hearing implant 150L. The time slot 471 is shifted or misaligned with about one-fourth of a length of one time slot relative to the time slots at the first processing unit 120L, e.g. master processing unit. This misalignment of time slots prevents the first hearing implant 150L from detecting the first synchronization marker 465a because the latter extends across two adjacent time slots at the first hearing implant 150L.

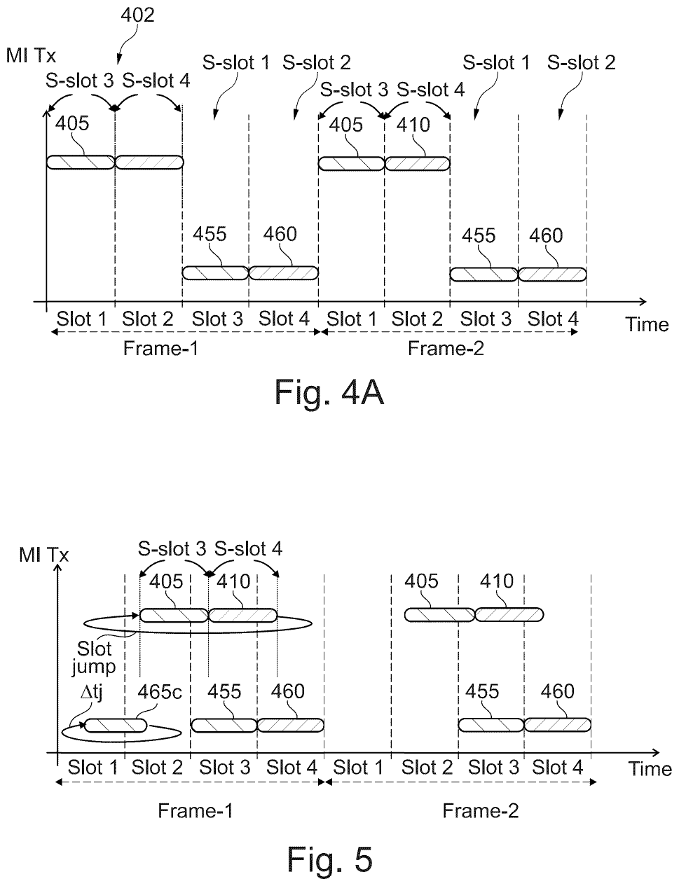

The first hearing implant 150L therefore advances or slides its time slots 1-4, such as the exemplary time slot 471, with predetermined time steps between frames of the plurality of consecutive frames using a wrap-around scheme as schematically indicated by “Time slide” arrow. A predetermined time step is preferably much shorter than a length of one time slot such as less 5% or less than 1% of the length of one time slot. The first hearing implant 150L continues to slide its time slots 1-4 until the latter time slots are sufficiently aligned with the time slots 1-4 at the first processing unit 120L to detect receipt of the first synchronization marker 465a at the first hearing implant 150L. The first control unit 108L of the first implant 150L may check the unique pair ID of a received synchronization marker to ensure it is transmitted from a paired hearing device, i.e. of the first hearing device 100L in the present situation. The first control unit 108L may reject the received synchronization marker if it is not transmitted by the paired device. In the latter situation, the first control unit 108L may thereafter continue the search for the first synchronization marker 465a. When the first synchronization marker 465a is detected, the first hearing implant 150L responds by reading a slot indicator bit or field of the first synchronization marker 465a as discussed in further detail below. The first hearing implant 150L may proceed, based on that time slot identification, to align time slots at the first hearing implant 150L, e.g. at the first control unit 108L, with the corresponding time slots at the first processing unit 120L of the first hearing device 100L. Hence, in effect synchronize data packet exchange or communication between the first hearing implant 150L and the first processing unit 120L through the first link 112L by aligning time slots 1-4 at the first processing unit 120L with the time slots 1-4 at the first control unit 108L. The first control unit 108L may proceed by shifting to a transmission mode and transmit an acknowledge message (ACK) 481, e.g. second acknowledge message, to the first processing unit 120L in time slot 4 at the first processing unit 120L as schematically illustrated by FIG. 4. Thereafter, the first processing unit 120L may respond by terminating/completing the acquisition of the first link 112L because the latter is now appropriately connected between the first processing unit 120 and the first hearing implant 150L in accordance with the first and second embodiments of the common communication protocol of the binaural hearing system 125. The first processing unit 120L and the first hearing implant 150L are accordingly ready to exchange the first ipsilateral data packets 455, 460 using time slots 3 and 4 as outlined in further detail below in connection with FIG. 4A.

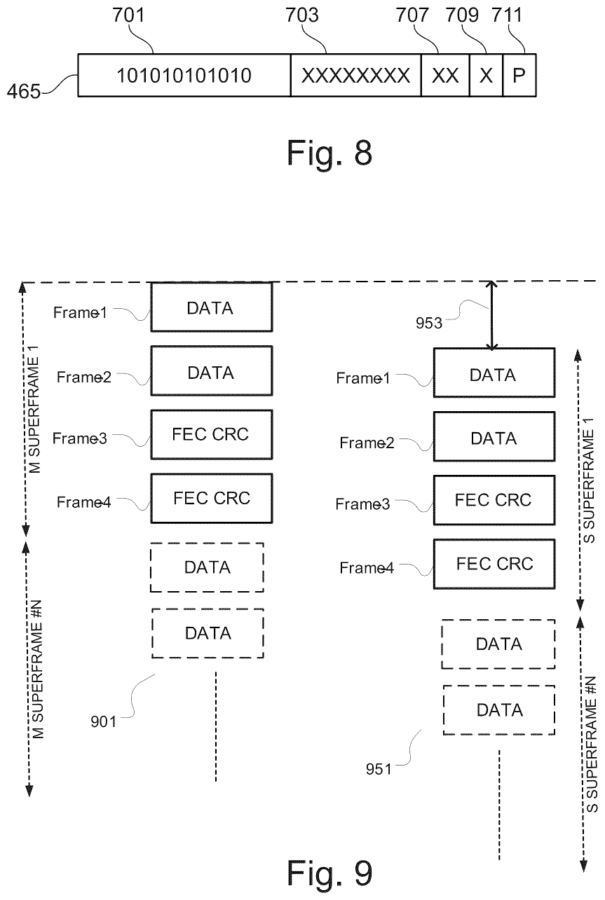

The skilled person will appreciate that the acquisition of the second link 112R of the exemplary binaural hearing system 125 may follow a corresponding scheme to create an appropriate connection between the second processing unit 120R and the second hearing implant 150R. Preferably, the second processing unit 120R operates as a master unit towards the second hearing implant 150R. However, a second synchronization marker (not shown), that has different unique pair ID to that of the first synchronization marker 465a, is preferably utilized by the second processing unit 120R and the second hearing implant 150R for the acquisition of the second link 112R. The difference between the unique pair IDs (e.g. as stored in the unique pair ID field 703 on FIG. 8) of the first synchronization marker 465a and the second synchronization marker ensures that only the appropriate pair of the first processing unit 120L, and hence the first hearing device 100L, and the first hearing implant 150L is connected to each other. Likewise, such that only the appropriate pair of the second processing unit 120R, and hence the second hearing device 100R, and the second hearing implant 150R is connected to each other. Otherwise, communication crosstalk for example between the first hearing device 100L and the second hearing implant 150R or vice versa could lead to erroneous pairing. The first synchronization marker 465a and the second synchronization marker may be distinguished by their unique pair IDs as discussed in further detail below.

FIG. 4A illustrates in schematic form exchange of first and second ipsilateral data packets 455, 460, 405, 410, respectively between the first and second hearing devices 100L, 100R and their associated hearing implants 150L, 150R in accordance with the embodiments of the common communication protocol of the binaural hearing system 125. The illustrated exchange of the first and second ipsilateral data packets 455, 460, 405, 410, respectively, is carried out after the first processing unit 120L has acquired the first bidirectional wireless communication link to make the connection, e.g. first connection, between the first hearing device 100L and the first hearing implant 150L as outlined in detail above. The second processing unit 120R has independently, and for example substantially simultaneously, acquired the second bidirectional wireless communication link 112R as outlined in detail above to make the connection, e.g. a second connection, between the slave/second hearing device 100R and the second hearing implant 150R. During the acquisition of the first link 112L the first processing unit 120L is preferably configured to operate as a master towards the first hearing implant 150L. The respective acquisitions of the first link 112L and second link 112R that establish respective connections through the first and second links 112L, 112R may be viewed as an intermediate step of the acquisition sequence in accordance with the embodiments of the common communication protocol. The subsequent acquisition of the bilateral link 114 that establishes a connection between the first processing unit 120L and the second processing unit 120R can be viewed as a final step of the acquisition sequence. The acquisition of the first link 112L may establish the connection between the first processing unit 120L and the first hearing implant 150L by aligning, e.g. synchronizing, the plurality of time slots at the first processing unit 120L with the corresponding time slots at the first hearing implant 150L. Likewise, the acquisition of the second link 112R may establish the connection between the second processing unit 120R and the second hearing implant 150R by aligning, e.g. synchronizing, the plurality of time slots at the second processing unit 120R with the corresponding time slots at the second hearing implant 150R. The acquisition of the bilateral link 114 may establish the connection between the first and second processing unit 120L, 120R, and hence between the first and second hearing devices 100L, 100R, by aligning, e.g. synchronizing, the plurality of time slots at the first processing unit 120L with the corresponding time slots at the second processing unit 120R. Consequently, after completion of the acquisition sequence and entry into normal operation of the binaural hearing system 125, the exchange of respective data packets between the first processing unit 120L and the first hearing implant 150L, between the second processing unit 120R and the second hearing implant 150R and between the first and second processing units 120L, 120R may be synchronized. This feature inter alia reduces power consumption and/or supports robust real-time transmission of the digital audio signals of the binaural hearing system 125.

In connection with the intermediate step of the acquisition sequence, FIG. 4A illustrates the exchange of the first ipsilateral data packets 455, 460 between the first processing unit 120L and the first hearing implant 150L. In a similar manner the second ipsilateral data packets 405, 410 are exchanged between the second processing unit 120R and the second hearing implant 150R. In a frame, like frame-1, the first processing unit 120L transmits the first ipsilateral data packets 455, 460 to the first hearing implant 150L using respective predetermined time slots, such as slots 3 and 4, or any other pair, of the illustrated time slots 1-4, at the first processing unit 120L. The first processing unit 120L transmits the first ipsilateral data packets 455, 460 in respective ones of the plurality of consecutive frames such as frame-1, frame-2 etc. The master side of the binaural hearing system 125 may be seen as a combination of the first hearing device 100L and the first hearing implant 150L. The slave side of the binaural hearing system 125 may be seen as a combination of the second hearing device 100R and the second hearing implant 150R.

The second processing unit 120R exchanges the second ipsilateral data packets 405, 410 with the second hearing implant 150R of the binaural hearing system 125 using respective predetermined time slots, such as time slots 3 and 4, at the slave side of the binaural hearing system, such as at the second processing unit 120R, following a similar transmission scheme and data packet structure as that of the first processing unit 120L.

The skilled person will understand that other embodiments of the common communication protocol may specify more than four time slots per frame such as between 5 and 16 time slots. Irrespective of the actual number of time slots, such as at least four time slots, these are preferably non-overlapping time slots. Each time slot may have a length between 24 μs and 384 μs for example depending on a number of bits of at least some of the exchanged data packets, such as the first, second and bilateral data packets. The length of each of the time slots may be identical. Each of the first ipsilateral data packets 455, 460 and the second ipsilateral data packets 405, 410 may comprise identical number of bits for example between 4 bits and 96 bits such as between 6 bits and 24 bits. The number of bits in a particular data packet may depend on its type of content, i.e. digital audio signals, control information or a combination of both.

At least some of the first ipsilateral data packets 455, 460 and at least some of the second ipsilateral data packets 405, 410 comprise digital audio signals and/or control information or data. The control information may be held in a packet header and the digital audio signals held in a packet payload as described in further detail below in connection with FIG. 8. The digital audio signals may be perceptually encoded to reduce the amount of audio data in the messages, such as the first ipsilateral data packets 455, 460 and second ipsilateral data packets 405, 410, for transmission. The respective digital audio signals of the first ipsilateral data packets 455, 460 and second ipsilateral data packets 405, 410 may be generated by, or derived, from respective first and second microphone arrangements 124L, 124R of the first and second hearing devices 100L, 100R. The first and second microphone arrangements 124L, 124R may be integrated with respective housings of the first and second hearing devices 100L, 100R. The respective digital audio signals of the first ipsilateral data packets 455, 460 and second ipsilateral data packets 405, 410 may alternatively be derived from remote microphone arrangements wirelessly coupled to the first and second hearing devices 100L, 100R. The respective digital audio signals may comprise sound such as speech, noise or any mixture thereof, for example sound present in a user's external environment or sound streamed sound from an audio enabled portable device. In some embodiments of the binaural hearing system 125, the first and second ipsilateral data packets 460, 410, respectively, that are exchanged in respective time slots 4 are transmitted from the first and second hearing implants 150L, 150R to their respective first and second processing units 120L, 120R in accordance with the common communication protocol. In that embodiment, the first processing unit 120L operates in receipt mode in time slot 4 and monitors the first link 112L to detect the first ipsilateral data packet 455. The slave/second processing unit 120R operates in a corresponding manner for receipt of the data packet 410 transmitted by the second hearing implant 150R in time slot 4 at the second processing unit 120R. The respective first and second ipsilateral data packets 460, 410, transmitted by the first and second hearing implants 150L 150R in their respective time slots 4, may exclusively comprise control information, i.e. no digital audio signals. Hence, data packets that exclusively comprise control information, i.e. control data, may be viewed as control data packets. The control information may include certain types of protocol parameters of the common data communication protocol such as pair ID information, i.e. the unique pair ID, error detecting codes etc. associated with the first hearing implant 150L and/or the second hearing implant 150R.

In some embodiments of the binaural hearing system 125 both of the first ipsilateral data packets 455, 460 are transmitted in time slots 3 and 4, respectively, from the first processing unit 120L to the first hearing implant 150L in accordance with the common communication protocol. In that embodiment, the first processing unit 120L operates in transmission mode (Tx) in both time slots 3 and 4 while the first hearing implant 150L simultaneously operates in receipt mode (Rx) in time slots 3 and 4 and monitors the first link 112L to detect each of the first ipsilateral data packets 455, 460. The second processing unit 120R and second hearing implant 150R operate in a corresponding manner for transmission of the second ipsilateral data packets 410, 410 and receipt thereof at the second hearing implant 150R, and at its second processing unit 120R. Further, in some embodiments of the common communication protocol, the type of content of the first and second ipsilateral data packets 455, 460, 405, 410, respectively, differs from frame to frame of the plurality of consecutive frames. The plurality of consecutive frames may optionally be organised in so-called super-frames which each comprises a plurality of individual frames, such as between 4 and 32 individual frames, to provide different types of content of the first and second ipsilateral data packets 455, 460, 405, 410, respectively, as discussed in additional detail below.

The skilled person will appreciate that the illustrated exemplary timing on FIG. 4A of the respective transmissions of the first and second ipsilateral data packets 455, 460, 405, 410 through the first and second links 112L, 112R, respectively, are worst-case misaligned or asynchronous. In this worst-case misalignment situation the time slots 3 and 4 at the second processing unit 120R are aligned with time slots 1 and 2 at the first processing unit 120L, as indicated by reference symbol 402 and S-slot 3 and S-slot-4, after acquisition of the first and second links 112L, 112R. In this worst-case alignment situation there are not any unoccupied time slots in a frame for data packet transmission during the acquisition of the bilateral link 114 or for exchange of the bilateral data packets during normal operation of the binaural hearing system 125. The skilled person will appreciate that after the acquisitions of the first and second links 112L, 112R, respectively, the alignment between the plurality of time slots, e.g. slots 1-4, at the first processing unit 120L and the corresponding time slots at the second processing unit 120R will be random. Hence, a plurality of time slots may be misaligned with any amount for example by 0.5 slot, 1 time slot, 1.5 time slot and even as the illustrated worst-case situation with a misalignment of exactly two time slots out of the four time slots 1-4. This random time slot alignment is caused by the free-running clock generators and clock signals at the first and second processing units 120L, 120R. In practice the clock signals of the first and second processing units 120L, 120R, respectively, will differ slightly in frequency and phase even when the clock generators are nominally identical. Hence, the time slots at the master side, such as at the first processing unit 120L, and the slave side, such as at the second processing unit 120R, are most likely more or less unaligned, e.g. misaligned or asynchronous, after the intermediate step of the acquisition sequence of the binaural hearing system.

FIG. 5 illustrates in schematic form one embodiment, e.g. first embodiment, of the acquisition of the bilateral link 114 by the first processing unit 120L, operating as the master processing unit, and the second processing unit 120R be configured as a slave processing unit. The acquisition of the bilateral link 114 is carried out in response to, and hence after, the above-outlined acquisition of the first link 112L and the acquisition of the second link 112R and further in accordance with the acquisition sequence of the common communication protocol. The second processing unit 120R may enter into a listening mode or state in the unoccupied time slots 1 and 2 at the second processing unit 120R after the acquisition of the second link 112R. The first processing unit 120L may enter into a transmit mode or state in the unoccupied time slots 1 and 2 at the first processing unit 120L after the acquisition of the first link 112R. The first processing unit 120L may thereafter start to transmit the third synchronization marker 465c in the unoccupied time slots via the bilateral link 114 for detection by the second processing unit 120R as described in additional detail below.

As schematically illustrated, the second ipsilateral data packets 405, 410 in time slots 3 and 4, respectively, at the master side, such as at the first processing unit 120L, and the time slots 3 and 4 at the slave side, such as at the second processing unit 120R, are shifted or misaligned with about two-thirds of the length of one time slot. This situation is illustrated by indications of time slots 3 and 4, S-slot 3, S-slot 4, at the second processing unit 120R taking as reference the time slots 1-4 at the first processing unit 120L depicted on the time-axis. Since the first and second links 112L, 112R are established, the exchange of the first ipsilateral data packets 455, 460 at the master side, such as at the first processing unit 120L, and the exchange of the second ipsilateral data packets 405, 410 at the slave side, such as at the second processing unit 120R, through the connections made by the first and second links 112L, 112R, provide the user of the binaural hearing system with sound in at least one, and possibly, both ears during the subsequent acquisition of the bilateral link 114. The latter property is desirable because the transmission of the sound in at least one of the user's ears provides the user with basic auditory awareness to hear speech and other sound signals in the surrounding environment.

The subsequent acquisition of the bilateral link 114, and subsequent exchange of the bilateral data packets during connection, may further enhance the user's listening comfort and improve speech intelligibility by applying sophisticated binaural processing algorithms like beamforming, noise reduction etc. by the first and second processing units 120L, 120R, respectively.

For the purpose of acquiring the bilateral link 114, the second processing unit 120R is configured to shift, e.g. advance, transmission of the second ipsilateral data packets 405, 410 at the second processing unit 120R with one time slot at every predetermined number of frames of the plurality of consecutive frames for example every 8^{th}, 16^{th}, 32^{nd} or 64^{th} frame. The time slot shift of transmission of the second ipsilateral data packets 405, 410 uses a wrap-around scheme as illustrated by the depicted arrow “slot jump”. Hence, the shift of time slots at the second processing unit 120L frees different time slots over time at the second processing unit 120 for possible transmission or receipt of the bilateral data packets. As illustrated on FIG. 5, time slot 1 of frame-1 and frame-2 etc. is unoccupied or vacant at the current moment in time. The first processing unit 120L may be configured to concurrently generate and

transmit the third synchronization marker 465c to the second processing unit 120R through the bilateral link 114 using unoccupied time slots 1 and 2 at the first processing unit 120L. The first processing unit 120L is additionally configured to shift, e.g. slide, transmission of the third synchronization marker 465c with a predetermined time step through time slots 1 and 2 from frame to frame using a wrap-around scheme as schematically illustrated by arrow Δtj. The wrap-around scheme means that the sliding of the third synchronization marker 465c jumps back to the start of time slot 1 when the end of time slot 4 is reached by the sliding of the third synchronization marker 465c.

The predetermined time step is preferably much smaller than the slot length and hence also the frame length. The predetermined time step Δtj may be 250 ns for a frame length of 192 μs. The same ratio between the predetermined time step and the frame length may be used for alternative frame lengths for example frame lengths between 100 and 400 μs. The corresponding length of a time slot may be about 48 μs for a frame length of 192 μs where the common communication protocol allocates four time slots of the same length per frame as the present embodiment.

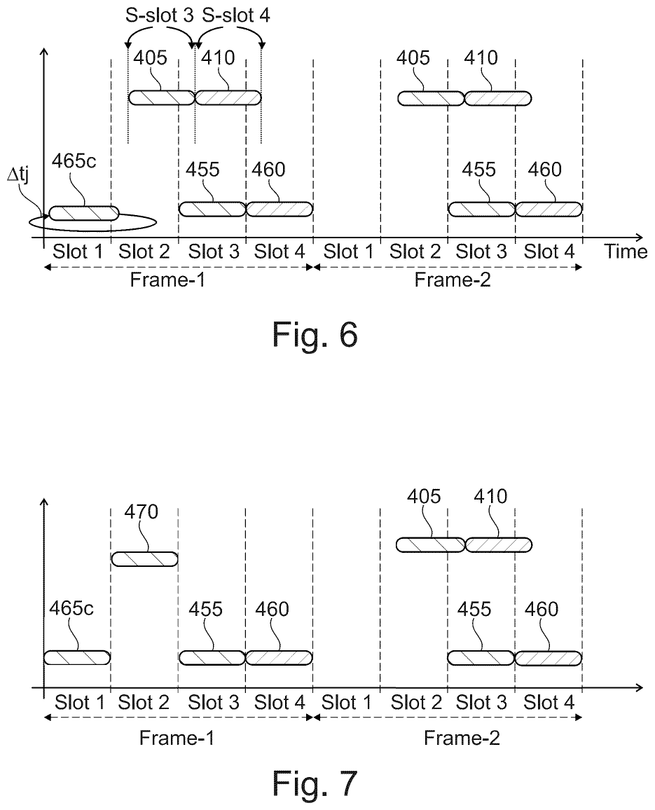

FIG. 6 illustrates in schematic form additional steps of the acquisition of the bilateral link 114 by the first processing unit 120L and second processing unit 120R according to the previously disclosed first embodiment of the binaural hearing system 125. The second processing unit 120R is configured to monitor the bilateral bidirectional wireless communication link 114 for the third synchronization marker 465c transmitted by first processing unit 120L as outlined above. In response to detection of the third synchronization marker 465c the second processing unit 120R interrupts the time slot hopping or shifting discussed above in connection with FIG. 5. The second processing unit 120R further responds by temporarily disconnecting or breaking the second link 112R. Accordingly, the second ipsilateral data packets 405, 410 are temporarily no longer transmitted by the slave side, such as the second processing unit 120R, of the binaural hearing system 125 which leaves all four time slots unoccupied by the second ipsilateral data packets 405, 410 at the slave side, such as the second processing unit 120R, as schematically depicted on FIG. 7. However, the first processing unit 120L continues to exchange the first ipsilateral data packets 455, 460 in time slots 3 and 4 whereby sound may be transmitted to the user's first side hearing implant 150L in an uninterrupted manner also during the temporary breakage of the second link 112R during the present embodiment of the acquisition sequence and binaural hearing system 125.

The first processing unit 120L preferably continues to transmit the third synchronization marker 465c while sliding the third synchronization marker 465c through time slots 1 and 2 using the wrap-around scheme until such time that the third synchronization marker 465c is arranged within time slot 1 and transmitted therein. The second processing unit 120R monitor for and detects the third synchronization marker 465c which may follow the above-outlined two-step scheme where the second processing unit 120R initially recognizes a predetermined search sequence 701 (refer to FIG. 8) and thereafter performs the “long packet search” to detect the entire content of the third synchronization marker 465c. The second processing unit 120R thereafter reads a slot indicator 709 (refer to FIG. 8) of the third synchronization marker 465c and aligns the time slots 1-4 at the second processing unit 120R with the corresponding time slots at the first processing unit 120L to synchronize the time slots, and hence the frames as well, at the first processing unit 120L and second processing unit 120R of the binaural hearing system 125. The second processing unit 120R proceeds by transmitting an acknowledge message (ACK) 470, e.g. a first acknowledge message, (refer to FIG. 7) back to the first processing unit 120L via the bilateral link 114 as schematically depicted on FIG. 8. The first processing unit 120L responds by completing the acquisition of the bilateral link 114 and the second processing unit 120R proceeds by re-establishing the connection through the second link 112R which has been temporarily interrupted. Thereafter, the first processing unit 120L responds by completing the acquisition sequence because each of the first link 112L, the second link 112R and the bilateral link 114 is now appropriately connected and the respective exchanges of data packages through these links 112L, 112R, 114 time aligned or synchronized in accordance with the exemplary embodiments, such as the first embodiment, of the common communication protocol of the binaural hearing system 125.

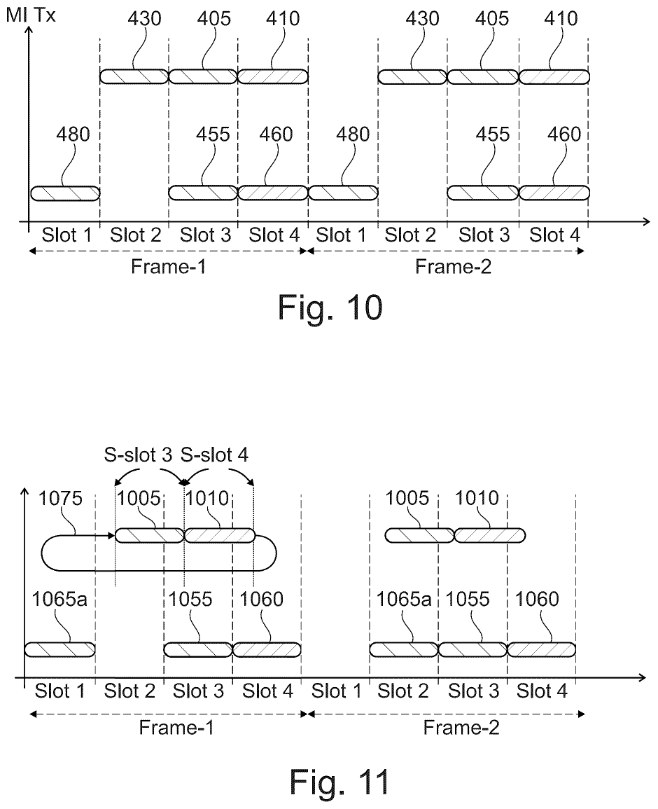

In response to completion of the acquisition sequence, the first processing unit 120L, the second processing unit 120R, the first hearing implant 150L and the second hearing implant 150R enter normal operation, i.e. a “payload mode”, where the first and second ipsilateral data packets 455, 460, 405, 410, respectively, are exchanged in time slots 3 and 4. The bilateral data packets 480, 430 are further exchanged in time slots 1 and 2, respectively, of the consecutive frames as schematically illustrated on FIG. 10.

Some embodiments of the common communication protocol comprise that the first processing unit 120L is configured to respond to receipt of the first acknowledge message (ACK) 470 by starting a countdown sequence before the payload mode is started. The count-down sequence may comprise an exchange of several rounds, e.g. 2, 3 or 4 rounds, of the third synchronization marker 465c and accompanying first acknowledge message (ACK) 470 between the first processing unit 120L and the second processing unit 120R before the completion of the acquisition sequence and entry into the payload mode. This countdown sequence may be advantageous to further verify the reliability of the bilateral link 114.

After the acquisition of the bilateral link 114, the second processing unit 120R re-connects the temporarily disconnected second link 112R so that the exchange of the second ipsilateral data packets 405, 410 between the second hearing device 100R and second hearing implant 150R is restarted.

The skilled person will appreciate that the uninterrupted operation of at least one of the first and second links 112L, 112R, respectively, during the entire acquisition sequence is a favourable property. This uninterrupted operation is carried out by the on-going exchange of the first and second ipsilateral data packets 455, 460, 405, 410, respectively, so that sound pick-up at first and second hearing devices 100L, 100R is processed and conveyed to the user of the binaural hearing system 125.

FIG. 8 illustrates an exemplary synchronization marker 465 that may be utilized by each of the first (465a), second (not shown) and third (465c) synchronization markers for the respective acquisitions of the bilateral link 114, the first link 112L and the second link 112R. The exemplary synchronization marker 465 preferably comprises a unique pair ID field 703. The unique pair ID field 703 may be utilized by the first, second and third synchronization markers for storage of respective unique pair IDs. The unique pair IDs may be a code or number and comprise between 8 bits and 32 bits. A first unique pair ID may be utilized to pair the first hearing device 100L and the first hearing implant 150L to each other. The first unique pair ID may be stored in a memory of the first processing unit 120L and further stored in a memory of the first control unit 108L of the first hearing implant 150L. The first unique pair ID may for example be stored during manufacture of the first hearing device 100L or later in connection with a fitting of the binaural hearing system 125 to the user. The first unique pair ID may be stored in a similar manner in the memory of the first control unit 108L. A second unique pair ID may be utilized to pair the second hearing device 100R and the second hearing implant 150R to each other. The second unique pair ID may be stored in the memory of the second processing unit 120R and further stored in a memory of the second control unit 108R of the second hearing implant 150R. The second unique pair ID may for example be stored in the second processing unit 120R and the second control unit 108R in a similar manner to the first unique pair ID as outlined above.

A third unique pair ID may be utilized to pair the first hearing device 100L and the second hearing device 100R to each other. The third unique pair ID may be stored in the memory of the first processing unit 120L and further stored in the memory of the second processing unit 120R. The third unique pair ID may for example be stored in the second processing unit 120R and the first processing unit 120L in a similar manner to the first unique pair ID as outlined above.

The different unique pair IDs ensure that only the appropriate pair of hearing devices and hearing implants are connected to each other during the respective acquisitions of the first link 112L, second link 112R and the bilateral link 114. Otherwise, unintended crosstalk between the first and second hearing implants 150L, 150R of the binaural hearing system 125 could lead to erroneous pairing of the first and second hearing devices 100L, 100R and their respective first and second hearing implants 150L, 150R. The unique pair IDs may be effective to prevent such unintended crosstalk and potentially erroneous pairing of hearing devices of two different but nearby located binaural hearing systems.

The synchronization marker 465 can be viewed as a special type of data packet and may have the same length and number of bits as at least some of the first and second ipsilateral data packets. The synchronization marker 465 may further comprise a predetermined search sequence 701, for example a predetermined binary pattern like 10101010 or equivalently 01010101. The latter is known to both the first processing unit 120L and the second processing unit 120R a priori in a similar manner to the unique pair IDs. The predetermined binary pattern may be received and recognized by the second processing unit 120R to detect the synchronization marker 465 in a currently unoccupied time slot at the second processing unit 120R.

The synchronization marker 465 may comprise an optional wrap-around countdown number 707. The latter may be utilized by the first processing unit 120L and second processing unit 120R for providing an additional layer of safety once the second processing unit 120R and first processing unit 120L have acknowledged each other through the bilateral link 114 as described in additional detail below. A slot indicator 709 tells the second processing unit 120R in which time slot at the master side the synchronization marker 465 is arranged to allow the second processing unit 120R in aligning its time slots with the corresponding time slots at the first processing unit 120L. The second processing unit 120R may for example determine a time offset between the already known time slots at the second processing unit 120R and the corresponding time slots at the first processing unit 120L for alignment. Since, the length of a time slot, e.g. 48 μs, is known to the second processing unit 120R the time offset can indicate slot alignment.

A “long packet search” may be started by the second processing unit 120R in the time slot where the predetermined search sequence 701 was last observed. The long packet search is utilized by the second processing unit 120R to read and evaluate the entire data content of the third synchronization marker 465c once the predetermined binary pattern is recognized or detected by the second processing unit 120R. The third synchronization marker 465c may comprise a parity bit 711 or any other type of suitable error detection and/or correction code. The second processing unit 120R may utilize the error detection and/or correction code to evaluate data integrity of the synchronization markers in well-known manners.

FIG. 9 illustrates an exemplary super-frame structure where each super-frame comprises a plurality of individual frames, such as between 4 and 32 individual frames, of the plurality of consecutive frames in accordance with one embodiment of the common communication protocol. The frames at the first processing unit 120L of the binaural hearing system are structured in consecutive first super-frames 901 and the super-frames at the second processing unit 120R of the binaural hearing system are structured in consecutive second super-frames 951. Each frame such as frame-1 may carry the same type of data, for example digital audio signals, which means that all data packets of the frame in question hold the same type of data e.g. digital audio signals, control information or error-correction codes like CRC and/or forward error-correction codes (FEC) etc. The super-frames at the first and second processing units 120L, 120R, respectively, may be misaligned in time, i.e. a time off-set, as schematically illustrated by an off-set arrow 953. The second processing unit 120R may be configured to detect this time off-set and transmit it to the first processing unit 120L. The first processing unit 120L may proceed to align the first and second super-frames 901, 951 between the first processing unit 120L and second processing unit 120R.

FIGS. 11, 12 and 13 illustrate various steps of an acquisition sequence of an alternative, e.g. second, embodiment of the exemplary binaural hearing system 125 utilizing an alternative common communication protocol to establish connection through the bilateral link 114 between the first and second hearing devices 100L, 100R. The first hearing device 100L may be configured as a master and the second hearing device 100R may be configured as slave. The acquisition sequence additionally establishes respective connections through the first and second links 112L, 112R to the first and second hearing implants 150L, 150R.

The skilled person will understand that the configuration of the first and second hearing devices 100L, 100R as master and slave may be carried out in connection with manufacturing of the binaural hearing system 125. Alternatively, the configuration of the first and second hearing devices 100L, 100R as master and slave may be carried out in connection with a fitting procedure of the binaural hearing system 125 on the user for example by utilizing an appropriately programmed computer connected to the binaural hearing system 125.

The illustrated exchanges of the first and second ipsilateral data packets 1055, 1060, 1005, 1010, respectively, depicted on FIG. 11 are carried out after the first processing unit 120L has acquired the connection, e.g. a first connection, to the first hearing implant 150L through the first link 112L in accordance with the exemplary embodiments, such as the second embodiment, of the binaural hearing system 125 operating in accordance with a corresponding embodiment of the common communication protocol. The operation of the present alternative, or second, embodiment of the binaural hearing system 125 follows the steps of the above-described acquisition of the first link 112L in accordance with the first embodiment of the binaural hearing system 125 and the common communication protocol. The second processing unit 120R has independently, and for example substantially simultaneously, acquired the connection, e.g. a second connection, to the second hearing implant 150R through the second link 112R. The acquisition may follow the steps of the above-described acquisition of the first link 112L in accordance with the first embodiment. After, and in response to, the respective acquisitions of the first link 112L and second link 112R, the subsequent exchange of respective data packets between the first processing unit 120L and the first hearing implant 150L and between the second processing unit 120R and the second hearing implant 150R may be similar to the one described above in the first embodiment of the exemplary binaural hearing system 125.

However, as illustrated by FIG. 11, the subsequent acquisition of the bilateral link 114 carried out by the first processing unit 120L and the second processing unit 120R in accordance with the second embodiment comprises inter alia a transmission of a first synchronization marker 1065a in a fixed predetermined time slot at the first processing unit 120L, e.g. slot 1, other than time slots already occupied by the first ipsilateral data packets 1055, 1060 as illustrated. The first synchronization marker 1065a may be transmitted in each frame, or a subset of frames e.g. every 4th or 8th frame etc., of a plurality of consecutive frames, i.e. frame-1, frame-2 etc.

Concurrently, the second processing unit 120R advances or slides transmission of the second ipsilateral data packets 1005, 1010 with predetermined time steps between frames of the plurality of consecutive frames using a wrap-around scheme. The predetermined time step is preferably shorter than a length of one time slot such as less than 5% or less than 1% of the length of one time slot. The second processing unit 120R may advance or slide the transmission of the second ipsilateral data packets 1005, 1010 for two or more round trips through time slots 1-4 in a first time direction such as from slots 2 and 3 towards slots 3 and 4. This advancement of the transmission of the second ipsilateral data packets 1005, 1010 is schematically depicted on FIG. 11 by wrap-around arrow 1075. The wrap-around scheme means that that the sliding of the time slots at second processing unit 120R, such as S-slot 3 and S-slot-4, jumps back to time slot 1 when the end of time slot 4 is reached by the sliding of the second ipsilateral data packets 1005, 1010.

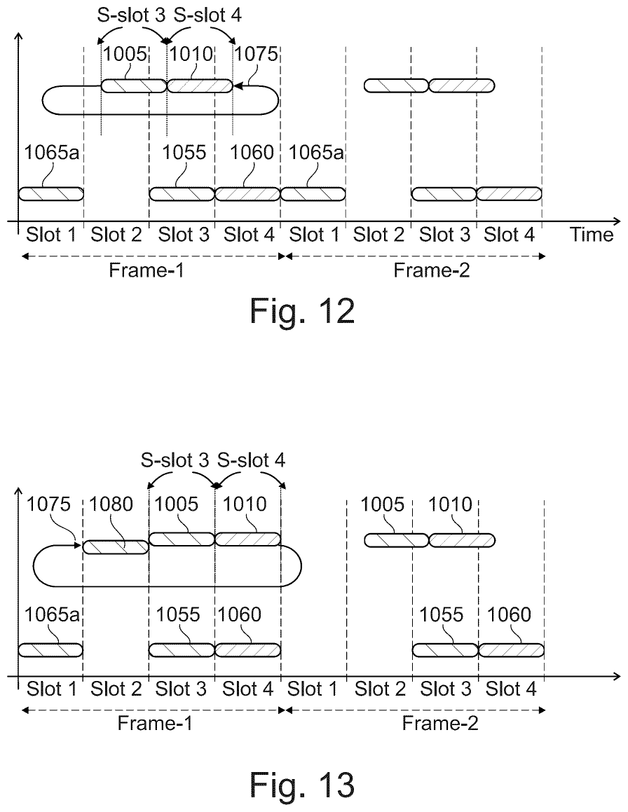

The second processing unit 120R may thereafter temporarily interrupt advancing the transmission of the second ipsilateral data packets 1005, 1010 for a predetermined time period but continue transmission of the second ipsilateral data packets 1005, 1010. Subsequently, the second processing unit 120R may advance the transmission of the second ipsilateral data packets 1005, 1010 for two or more round trips through time slots 1-4 in a second time direction, opposite to the first time direction, such as from slots 2 and 3 towards slots 1 and 2. This opposite advancement or sliding of the transmission of the second ipsilateral data packets 1005, 1010 is schematically depicted on FIG. 12 by an oppositely oriented wrap-around arrow 1075. The second processing unit 120R further monitors the bilateral link 114 during the bi-directional time wise advancement of the second ipsilateral data packets 1005, 1010 to detect receipt of the first synchronization marker 1065a. The skilled person will understand that this opposite time-wise sliding of the transmission of the second ipsilateral data packets 1005, 1010 is advantageous to compensate for the previously discussed free-running clock generators and clock signals at the first and second processing units 120L, 120R, respectively. Hence, the opposite time-wise sliding of the transmission of the second ipsilateral data packets 1005, 1010, respectively, guarantees alignment when the respective clock signals at the first and second processing units 120L, 120R differ in frequency.

As discussed above, the time slots at the master side, such as the first processing unit 120L, and the time slots at the slave side, such as the second processing unit 120R, are most likely more or less misaligned, or asynchronous, after the intermediate step of the acquisition sequence of the binaural hearing system 125 as illustrated by FIG. 12 where the time slots 3 and 4 at the first processing unit 120L and time slots 3 and 4 at the second processing unit 120R are shifted or misaligned with about two-thirds of the length of one time slot.

The first processing unit 120L monitors the bilateral link 114 for an acknowledge message (ACK) 1080 transmitted by the second processing unit 120R in time slot 2 in response to detection of the first synchronization marker 1065a transmitted by the first processing unit 120L in time slot 1 as schematically depicted on FIG. 13. The second processing unit 120R may check the unique pair ID of a received synchronization marker to ensure it is transmitted from a paired device, i.e. the first hearing device 100L in the present situation. The second processing unit 120R may reject the synchronization marker if it is not transmitted by the paired device. If the second processing unit 120R fails to detect the proper unique pair ID of the received first synchronization marker 1065a during the bi-directional time slide process of the second ipsilateral data packets 1005, 1010, the second processing unit 120R may be configured to re-start and repeat the time slide process until the first synchronization marker 1065a is detected. The second processing unit 120R finally interrupts the time slide transmission of process in response to its transmission of the acknowledge message (ACK) 1080 that confirms to the first processing unit 120L the second processing unit's 120R detection of the first synchronization marker 1065a. The first synchronization marker 1065a preferably comprises the previously discussed slot indicator 709 where the first processing unit 120L indicates to the second processing unit 120R which time slot at the master side, such as at the first processing unit 120L, that holds the third synchronization marker 465c so that the second processing unit 120R may align its time slots 1-4 with the corresponding time slots at the first processing unit 120L.

The first processing unit 120L responds to receipt of the acknowledge message (ACK) 1080 by completing the acquisition of the bilateral link 114 and the second processing unit 120R proceeds in a similar manner by completing the acquisition of the bilateral link 114. Thereafter, the first processing unit 120L proceeds by completing the acquisition sequence because each of the first link 112L, the second link 112R and the bilateral link 114 is now appropriately connected and corresponding time slots of all the links are time aligned or synchronized in the binaural hearing system 125 in accordance with the common communication protocol. In response to completion of the acquisition sequence, the first processing unit 120L, the second processing unit 120R, the first hearing implant 150L and the second hearing implant 150R each commence normal operation, i.e. a “payload mode”. In the payload mode the first and second ipsilateral data packets 1055, 1060, 1005, 1010, respectively, are exchanged in time slots 3 and 4. The bilateral data packets 480, 430 are exchanged in time slots 1 and 2, respectively. The bilateral data packets 480, 430 are not depicted on FIG. 13 but are schematically illustrated on FIG. 10.

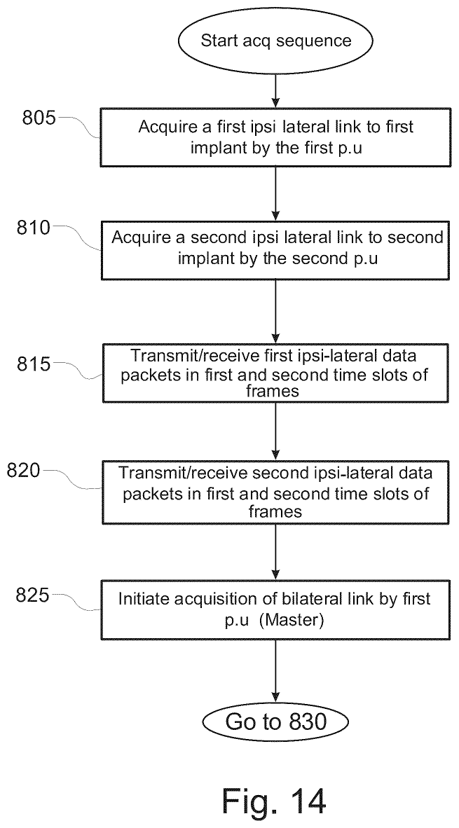

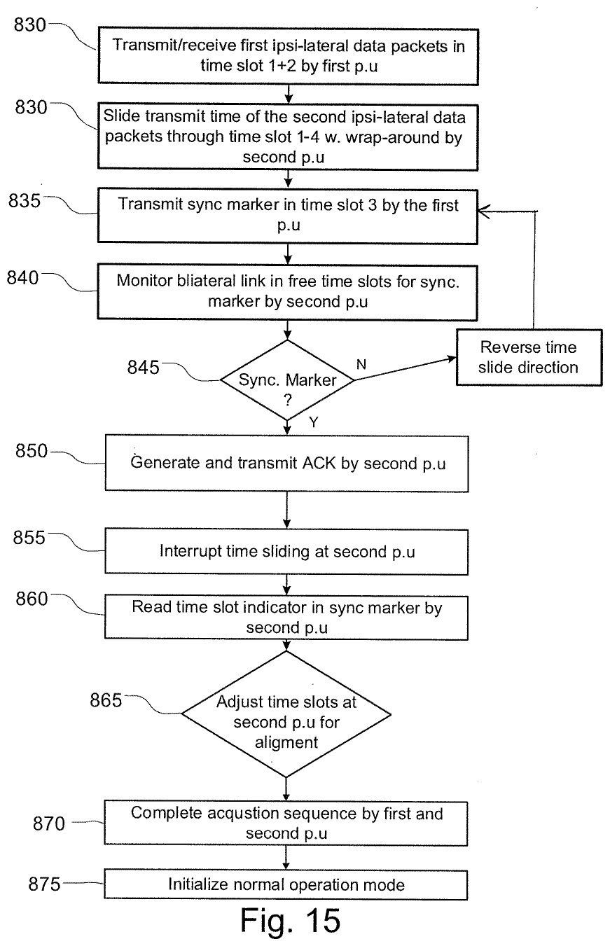

FIG. 14 and FIG. 15 are flow charts illustrating steps 805-875 carried out by the first and second processing units 120L, 120R, respectively, (“first p.u” and “second p.u”) of the first and second hearing devices 100L, 100R, respectively, in connection with the above-outlined acquisition of the bilateral bidirectional wireless communication link 114 according to the alternative embodiment of the exemplary binaural hearing system 125.

### LIST OF REFERENCE SYMBOLS

100L first/master hearing device
100R second/slave hearing device
102L first magnetic coil antenna of the first hearing implant
102R second magnetic coil antenna of the second hearing implant
104L first rechargeable battery assembly
104R second rechargeable battery assembly
106L first electrode array
106R second electrode array
107L first power line/wire
107R second power line/wire
108L first control unit
108R second control unit
110L first receiver (Rx) charging coil
110R second receiver (Rx) charging coil
112L first bidirectional wireless communication link/first link
112R second bidirectional wireless communication link/second link
114 bilateral bidirectional wireless communication link/bilateral link/third link
116L first magnetic coil antenna of the first hearing device
116R second magnetic coil antenna of the second hearing device
118L first transceiver
118R second transceiver
120L first/master processing unit
120R second/slave processing unit
122L first data bus
122R second data bus
124L one or more first microphones
124R one or more second microphones
125 binaural hearing system
126L first system clock generator
126R second system clock generator
128L first radio interface
128R second radio interface
130L first RF antenna
130R second RF antenna
150L first hearing implant
150R second hearing implant
250 user's head
402 reference symbol
405,410 second ipsilateral data packets
430, 480 bilateral data packets
455, 460 first ipsilateral data packets
465 synchronization marker
465a first synchronization marker
465c third synchronization marker
470 first acknowledge message (ACK)
471 time slot
481 second acknowledge message (ACK)
701 predetermined search sequence
703 unique pair ID field
709 slot indicator
707 countdown wrap-around counter
711 parity bit
805-875 processing steps
901 first super-frames
951 second super-frames
953 off-set arrow
1005, 1010 second ipsilateral data packets
1055, 1060 first ipsilateral data packets
1065a first synchronization marker (second embodiment)
1075 wrap-around arrow/oppositely oriented arrow
1080 acknowledge message (ACK)
Δtj predetermined time step

1. A binaural hearing system comprising:
a first hearing device, a second hearing device, a first hearing implant, and a second hearing implant;
wherein the first hearing device is communicatively connectable to the first hearing implant via a first bidirectional wireless communication link for exchange of first ipsilateral data packets;
wherein the second hearing device is communicatively connectable to the second hearing implant via a second bidirectional wireless communication link for exchange of second ipsilateral data packets;
wherein the first hearing device and second hearing device are communicatively connectable via a bilateral bidirectional wireless communication link for exchange of bilateral data packets;
wherein the bilateral bidirectional wireless communication link, and the first and second bidirectional wireless communication links are associated with a common communication protocol which comprises a plurality of consecutive frames;
wherein the binaural hearing system is configured to:
acquire the first bidirectional wireless communication link using at least a first processing unit of the first hearing device;
acquire the second bidirectional wireless communication link using at least a second processing unit of the second hearing device; and
acquire the bilateral bidirectional wireless communication link after the first and second bidirectional wireless communication links are acquired.

2. The binaural hearing system according to claim 1, wherein the binaural hearing system is configured to maintain at least one of the first bidirectional wireless communication link and second bidirectional wireless communication link while acquiring the bilateral bidirectional wireless communication link.

3. The binaural hearing system according to claim 1, wherein the binaural hearing system is configured to acquire the first bidirectional wireless communication link by:
transmitting a first synchronization marker from the first processing unit to the first hearing implant;
detecting the first synchronization marker at the first hearing implant;
synchronizing a plurality of time slots at the first hearing implant with a plurality of time slots at the first processing unit based on the first synchronization marker; and
transmitting an acknowledge message from the first hearing implant to the first processing unit.

4. The binaural hearing system according to claim 3, wherein the binaural hearing system is configured to acquire the second bidirectional wireless communication link by:
transmitting a second synchronization marker from the second processing unit to the second hearing implant;
detecting the second synchronization marker at the second hearing implant;
synchronizing a plurality of time slots at the second hearing implant with a plurality of time slots at the second processing unit based on the second synchronization marker; and
transmitting an acknowledge message from the second hearing implant to the second processing unit.

5. The binaural hearing system according to claim 4, wherein the binaural hearing system is configured to:
slide the plurality of time slots at the first hearing implant using a wrap-around scheme; and
slide the plurality of time slots at the second hearing implant using the wrap-around scheme.

6. The binaural hearing system according to claim 4, wherein the binaural hearing system is configured to acquire the bilateral bidirectional wireless communication link by:
transmitting a third synchronization marker from the first processing unit to the second processing unit;
detecting the third synchronization marker at the second processing unit;
synchronizing the plurality of time slots at the second processing unit with the plurality of time slots at the first processing unit based on the third synchronization marker; and
transmitting an acknowledge message from the second processing unit to the first processing unit.

7. The binaural hearing system according to claim 6, wherein the binaural hearing system is configured to acquire the bilateral bidirectional wireless communication link by:
time-shifting a transmission of the second ipsilateral data packets at the second processing unit; and
time-shifting a transmission of the third synchronization marker with a predetermined time step at the first processing unit.

8. The binaural hearing system according to claim 7, wherein the predetermined time step is less than 5% of a time slot length.

9. The binaural hearing system according to claim 4, wherein the binaural hearing system is configured to acquire the bilateral bidirectional wireless communication link by:
transmitting a third synchronization marker from the first processing unit to the second processing unit;
temporarily breaking the second bidirectional wireless communication link by the second processing unit;
detecting the third synchronization marker at the second processing unit;
transmitting an acknowledge message by the second processing unit to the first processing unit; and
re-acquiring the second bidirectional wireless communication link.

10. The binaural hearing system according to claim 9, wherein the binaural hearing system is configured to time-shift a transmission of the second ipsilateral data packets at the second processing unit, and time-shift a transmission of the third synchronization marker with a predetermined time step at the first processing unit; and
wherein the binaural hearing system is configured to acquire the bilateral bidirectional wireless communication link also by interrupting the time-shifting of the transmission of the second ipsilateral data packets while maintaining the time-shifting of the transmission of the third synchronization marker.

11. The binaural hearing system according to claim 3, wherein the binaural hearing system is configured to slide the plurality of time slots at the first hearing implant based on predetermined time steps using a wrap-around scheme.

12. The binaural hearing system according to claim 1, wherein the binaural hearing system is configured to maintain the first bidirectional wireless communication link and the second bidirectional wireless communication link while acquiring the bilateral bidirectional wireless communication link.

13. The binaural hearing system according to claim 1, wherein the binaural hearing system is configured to acquire the bilateral bidirectional wireless communication link by time-shifting a transmission of the second ipsilateral data packets at the second processing unit by a predetermined time step using a wrap-around scheme, wherein the predetermined time step is shorter than a time slot length.

14. The binaural hearing system according to claim 13, wherein the binaural hearing system is configured to time-shift the transmission of the second ipsilateral data packets by:
advancing the transmission for one or more round trips in a first time direction;
temporarily interrupting the advancement of the transmission for the one or more round trips; and
restarting the advancement of the transmission in a second time direction.

15. The binaural hearing system according to claim 3, wherein the first synchronization marker comprises:
a predetermined binary sequence, or
a time slot indicator for indicating a certain time slot holding the first synchronization marker.

16. The binaural hearing system according to claim 1, wherein each frame of the plurality of consecutive frames comprises at least four non-overlapping time slots.

17. The binaural hearing system according to claim 16, wherein a length of each of the at least four non-overlapping time slots is identical.

18. The binaural hearing system according to claim 1, wherein at least a subset of the first and second ipsilateral data packets comprises digital audio data.

19. The binaural hearing system according to claim 1, wherein at least a subset of the bilateral data packets comprises digital audio data.

20. The binaural hearing system according to claim 3, wherein the first synchronization marker comprises an ID for pairing the first hearing device and the first hearing implant.

21. A method performed by a binaural hearing system comprising a first hearing device, a first hearing implant, a second hearing device, and a second hearing implant, the method comprising:
acquiring a first bidirectional wireless communication link between the first hearing device and first hearing implant;
exchanging first ipsilateral data packets through the first bidirectional wireless communication link;
acquiring a second bidirectional wireless communication link between the second hearing device and second hearing implant;
exchanging second ipsilateral data packets through the second bidirectional wireless communication link;
acquiring a bilateral bidirectional wireless communication link between the first and second hearing devices, after the first and second bidirectional wireless communication links are acquired; and
exchanging bilateral data packets between the first hearing device and the second hearing device.
